(19) United States
(12) Patent Application Publication
(10) Pub. No.: US 2007/0129639 A1
(43) Pub. Date: Jun. 7, 2007

(54) Methods and analysis for cardiac ischemia detection

(76) Inventors: Hongxuan Zhang, Baltimore, MD (US); Nitish V. Thakor, Clarksville, MD (US); Jeffrey M. Wallace, Charlestown, RI (US); Ananth Natarajan, San Marino, CA (US)

Correspondence Address: KONRAD RAYNES & VICTOR, LLP, 315 S. BEVERLY DRIVE, # 210, BEVERLY HILLS, CA, 90212, US

(21) Appl. No.: 11/032,586

(22) Filed: Jan. 10, 2005

Related U.S. Application Data
(60) Provisional application No. 60/535,860, filed on Jan. 11, 2004.

(51) Int. Cl. A61B 5/04

(52) U.S. Cl. 600/509

(57) ABSTRACT

One embodiment relates to a method of monitoring the heart of a subject for evidence of at least one of myocardial ischemia and infarction (MI/I). The method includes sensing an intra-cardiac electrical signal from at least one lead positioned in a subject. The method also includes detecting MI/I using at least one of the QRS portion of the intra-cardiac electrical signal and the ST portion of the intra-cardiac electrical signal. The detecting MI/I includes using an integer coefficient filter to extract MI/I information from the intra-cardiac electrical signal.

This application claims priority in U.S. Provisional Application No. 60/535,860, filed Jan. 11, 2004, which is hereby incorporated by reference in its entirety.

### FIELD OF INVENTION

Embodiments relate to methods and apparatus for detection and treatment of the disease process known as myocardial ischemia and/or infarction (MI/I).

### BACKGROUND

Myocardial ischemia and/or infarction (MI/I) may be caused by a lack of blood, oxygen, and nutrients to the contractile heart cells. MI/I detection and analysis may be done by expert cardiologists manually or using a computer based algorithm to find the related ischemic changes within ECG signals. Moreover, many methods and algorithms have focused on changes of ST-segment or T wave.

### BRIEF DESCRIPTION OF THE DRAWINGS

Certain embodiments of the invention are described with reference to the accompanying drawings, which, for illustrative purposes, are not necessarily drawn to scale.

FIG. 1 illustrates a diagram and flowchart describing a preferred embodiment of a cardiac ischemia detection system.

FIG. 2 illustrates a flowchart of an implementation of a software detection system in accordance with a preferred embodiment.

FIGS. 3 (a) and 3 (b) illustrate a diagram of embodiments of an intra-cardiac lead system that can be used to detect cardiac ischemia, with FIG. 3 (a) illustrating an intra-cardiac bipolar lead system and FIG. 3 (b) illustrating an intra-cardiac unipolar lead system.

FIG. 4 (a) illustrates an intra-cardiac lead system in accordance with an embodiment, including a three lead mapping system modeled after Einthoven triangle leads I, II and III. FIG. 4 (b) illustrates an intra-cardiac lead system in accordance with an embodiment, including an augmented lead mapping system modeled after Einthoven triangle leads aVR, aVL and aVF.

FIG. 5 illustrates a flow chart of a MI/I detection strategy in accordance with one preferred embodiment.

FIG. 6 illustrates a flow chart of a preferred embodiment of a QRS wave detection strategy.

FIG. 7 illustrates a preferred embodiment of a QRS complex analysis based on continuous wavelet transforms.

FIGS. 8 (a) and 8 (b) illustrates discrete wavelet analysis based decomposition and Time Frequency Window Index (TFWI) calculation strategy in accordance with an embodiment of the present invention.

FIG. 9 illustrates an example of an embodiment of QRS detection of intra-cardiac data.

FIG. 10 illustrates an embodiment of Time Frequency Window Index changes that occur with ischemia via LAD occlusion.

FIG. 11 illustrates an example of methods comparison for ischemia detection in accordance with certain embodiments, including: ST segment, TFWI energy calculation, integer filter based intra-QRS energy (12-25 Hz), intra-QRS energy (25-40 Hz) and the identified occlusion event.

FIG. 12 illustrates an example to show stability of integer filter based intra-QRS energy (25-40 Hz) calculation for ischemia detection in accordance with certain embodiments of the present invention.

### DETAILED DESCRIPTION

Certain embodiments of the present invention relate to methods and approaches for analysis and/or detection of MI/I. Analysis and/or detection of MI/I includes identifying ischemia and/or infarction, if present. If present, the severity of such ischemia and/or infarction can also be determined.

Certain preferred embodiments relate to one or more intra-cardiac lead systems, strategies, and software or hardware based methods for MI/I diagnosis and detection. Intra-cardiac lead system implies that at least one of the electrodes in the system is within the cardiovascular system.

MI/I can be detected using implantable devices and methods according to the certain embodiments of the present invention. Embodiments may include a stand alone device or a modified version of any implantable device such as pacemaker, cardioverter, defibrillator, event recorder, loop recorder, etc. Certain preferred embodiments may include intra-cardiac catheter or lead and augmented lead system for unipolar or bipolar cardiac potential acquisition. Certain embodiments of present invention also relate to design, construction, placement and combination of intra-cardiac leads. Certain embodiments may also relate to design, construction and placement of a combination of electrodes in the thoracic cavity through subcutaneous or intrathoracic placement of one or more sensors. In one preferred embodiment, the electrodes form a configuration similar to the Einthoven triangle. The various leads may also use the can or the body of the implanted device as a sensor or circuit ground.

Embodiments may also include a series of methods based on spectrum, energy and their time and frequency distribution for MI/I features analysis and diagnosis. Embodiments may detect MI/I event utilizing different lead combinations and alert the patient using a variety of methods, including but no limited to vibration, electrical stimulation, and etc. The embodied algorithms may include signal analysis methods that work in time, frequency or joint time-frequency domain. The algorithms in certain preferred embodiments include time-frequency analysis, wavelet analysis, and digital filters. In the preferred embodiments, wavelet analysis may further includes continuous wavelet (CWT) and discrete wavelet (DWT) in one-dimension (1D), two-dimension (2D) and three dimension (3D) (corresponding to single or multiple features) for cardiac signal analysis. Based on wavelet and filter computation and analysis, certain embodiments utilize integer coefficient filters and quantized coefficient filters to reduce the computational load and make the algorithm suitable for microprocessors and microcontrollers used in implanted devices such as, but not limited to, pacemakers, cardioverters, defibrillators, event or loop recording devices. Certain embodiments may be utilized to detect MI/I from inside the body as compared with the traditional approach of detection by placing electrodes on the outer body surface of the torso.

Certain embodiments of the invention may also relate to detection of ischemic event including identifying particular features of the intra-cardiac signal. These features may include depolarization and repolarization. The methods in certain preferred embodiments may detect changes in depolarization and repolarization waves, especially in the composite QRS complex wave involving its full range of P-QRS-T wave morphology, in selected regions of heart, for the case of local MI/I or global MI/I in the whole heart. Certain embodiments can characterize the intra-cardiac signal in case of MI/I via signal shape changes or energy distribution changes, including temporal, spectral and combined approaches.

Certain embodiments may utilize, in an implantable device, one or more strategies with low computation resource requirements. The implantable device may incorporate elements including, but not limited to, a microprocessor, microcontroller, programmable logic device or programmable firmware to implement the algorithms and would provide automated diagnosis of any MI/I event. Embodiments may further include hardware, software or firmware modification of the aforementioned devices to have MI/I detection, altering and therapy initiating features.

Early recognition of symptomatic or asymptomatic myocardial ischemia (MI) that occurs during daily periods of rest and activity in patients may be helpful in preventing subsequent MI or fatal ischemic events. In addition, out-of-hospital monitoring may also help guide and control anti-ischemic therapy in these patients. Certain embodiments of the present invention describe methods including ECG detection lead systems and strategies for detecting of MI/I through one or more of the following methods: wavelet, time-frequency and band pass filter based QRS complex analysis.

In overview, certain preferred embodiments of the present invention include an ischemia detection strategy, which can be used for monitoring and detection tool for one or both of the following: surface ECG recordings and intra-cardiac ECG recordings. Certain embodiments of the invention can provide an easy and reliable means of discriminating between normal sinus rhythm and cardiac ischemia.

In certain embodiments, the ischemia detection strategy may be utilized independently to perform any single or combination of the following:
a. Qualitatively describe the heart rhythm, normal, ischemia and infarction.
b. Quantitatively diagnose the cardiac problem.
c. Easily integrate in pacemaker or other implantable cardiac monitoring instruments.
d. Detect ischemia as well as other heart problem, like ventricular tachycardia (VT), ventricular fibrillation (VF), etc.
e. Implanted devices and loop at event recoveries.
f. Implantable defibrillator and cardioverter.
g. Implanted thrombolytic drug delivery systems.
h. Event and alarm detection and feedback to the patient or the physician.

These and other features will be more fully understood from the following detailed description, which can be read in light of the accompanying drawings.

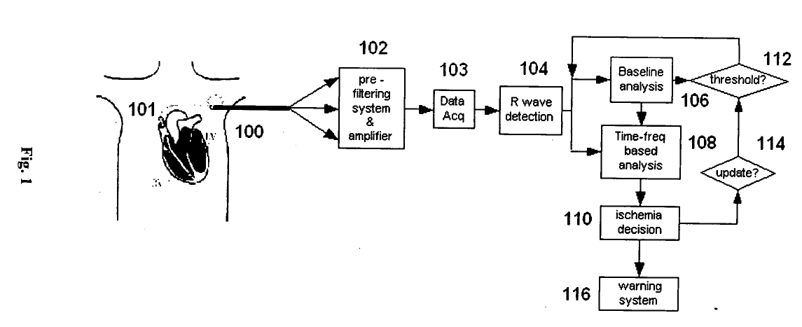

FIG. 1 describes the block and schematic diagram of an embodiment of an ischemia detection system. Based on the present clinical pacemaker leads system, an indwelling lead is typically inserted through the right atrium and ventricle. This lead 100 has either unipolar or bipolar sensors 101 on them. This embodiment can construct different cardiac potential vector systems, which allow for multiple lead intra-cardiac signals. The device 102 is a pre-filtering system which can be tuned to obtain better signal-to-noise (SNR) as well as a suitable signal range for data acquisition via adjusting the amplifier coefficients. Element 103 is a cardiac data acquisition device, in which the acquiring rate, data resolution and in/output methods can be controlled. Element 104 may perform R wave analysis and detection. In order to more accurately address the QRS complex during cardiac data acquisition, an improved strategy is employed in element 104, which can be more reliable in real time R-wave detection. Instances of this improved strategy can include: signal slope, amplitude and width of the QRS complex, also showing up as changes in the frequency or the spectrum of the QRS complex.

Element 104 represents computation in software or software. Certain embodiments are designed to discriminate between normal ECG and ischemic ECG through changes in the QRS complex. For embodiments involved in practical and clinical monitoring, the first step may preferably be to obtain baseline or normal data from each patient and use it as comparison for the discrimination of pathology. The baseline analysis 106 may be accomplished through the software and is used for making the threshold decision. The threshold should preferably be dynamic, as even in the same patient, during different stages, the heart works at different rhythm, which could affect the threshold level. In order to adapt to such variations, preferred embodiments include an algorithm having the flexibility to tune the warning threshold of ischemia or other cardiac pathology. Following 106, module 108 is a functional part that relates to time frequency joint analysis for ischemia information extraction. 110 is a detection module to determine MI/I if an ischemia or infarction event is present, and 116 is an alarm or warning system for notifying patient and/or doctor, and even transferring the ischemia alerting information. Module 112 and 114 are used to calibrate the threshold of MI/I detection and update it under different situations.

After obtaining the intra-cardiac data from module 100, 101, 102, 103 and R-wave detection 104, the embodiment may begin signal processing of the rhythm analysis.

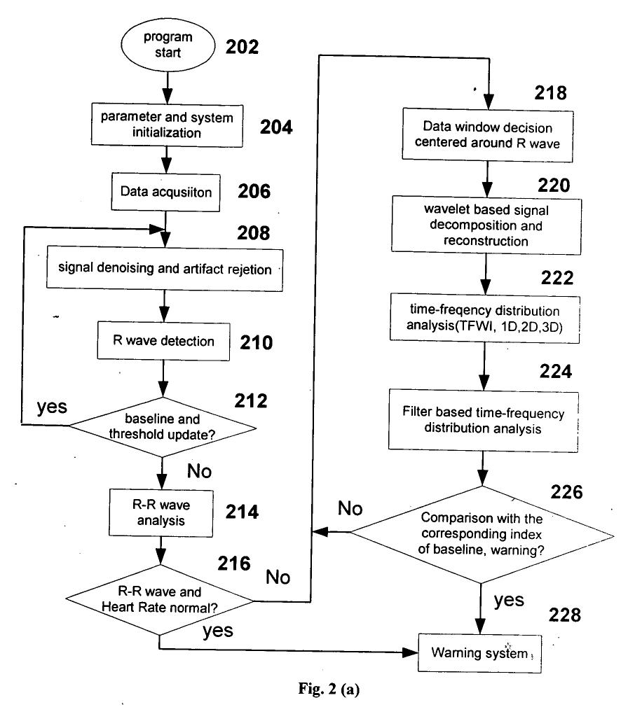

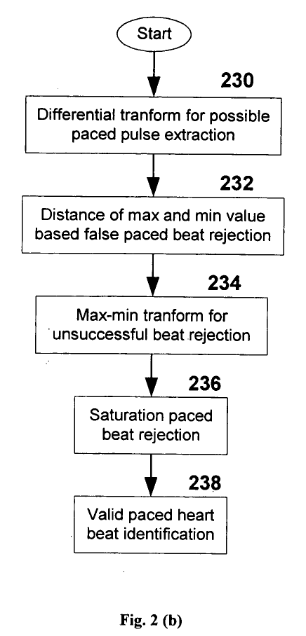

In FIG. 2, after block 202, parameter and system initialization 204 can be initiated and established. After the embodiment has been initialized, data can be acquired through the data acquisition 206 and begin to monitor the heart rhythm. Once the data has been acquired, raw data may go through denoising and artifact rejection 208, including but not limited to eliminating the 60 Hz interference, respiration, movement, other motion artifact, etc. After 208, cleaner cardiac data is separated according to R-R wave detection. This follows the embodiment's strategy based on MI/I detection from information extracted beat by beat. Function element 212 adapts each threshold and RR interval limit automatically. This adaptive approach may provide for an accurate use of intra-cardiac signals having many diverse signal characteristics, QRS morphologies, and heart rate changes. Accordingly, the RR wave detection and analysis 214 may also the basis for this embodiment.

Block 216 may be a rough detection function to discriminate various arrhythmias, including but not limited to ventricular tachycardia (VT), ventricular fibrillation (VF), and heart rate fluctuations. For general heart disease, when an early ischemia event does occur, the biological process may be very slow. For example, when there is an initial vessel occlusion, the general ECG may not discriminate the pathology immediately because ST change can be relatively small and slowly emerging. At this time, other features of the ECG signal may be responsive to ischemia related changes. With the help of signal processing methods, the present embodiment may demonstrate much better resolution, sensitivity and reliability. Such an embodiment may save much more time for the doctor and the patient by monitoring the early stage QRS complex changes resulting from MI/I. This strategy can be illustrated in elements 218, 220, 222, and 224. After R-wave detection, the cardiac data stream may be cut into short pieces based on beat interval or heart rate. A fixed window can be employed for each beat which is centered on R wave and whose width is tunable. The present embodiment including time-frequency filters and wavelet analysis focuses on the fixed window of each beat because time-frequency distribution changes can occur throughout the QRS-T complex once ischemia exists. The wavelet analysis may have 3 parts: one-dimension (1D, one parameter, such as the wavelet coefficients), two-dimension (2D, such as time-frequency distribution) and three-dimension (QRS energy distribution and time-frequency wavelet index (TFWI)). Element 226 is an ischemia decision judgment system for the calculation, comparison and detection. Element 228 is a warning part for signaling to the implanted device and then to the patient or the physician about the MI/I event.

Element 226, which may be based on time-frequency analysis, is an embodiment of a strategy and algorithm for detecting ischemia. This embodiment is computationally easier, more practical and suitable for many types of implanted devices, including but not limited to pacemakers, cardioverters, defibrillators, loop and event detectors. The filter based system in this embodiment can be accomplished by first placing a number of evenly spaced zeros around the unit circle (the zero of a digital filter is the value that makes the filter transfer function attenuate or go to the zero value). The zeros may have the net effects of attenuating the signal frequencies in the vicinity of the zero locations. Next poles are chosen (poles of a digital filter result high gain at that specific frequency). Poles may be placed on the unit circle to cancel some of the zeros. When a pole cancels a zero, the frequency corresponding to this location may no longer attenuate. Since each point on the unit circle is representative of frequency, the locations of poles and zeros determine the frequency response of the filter.

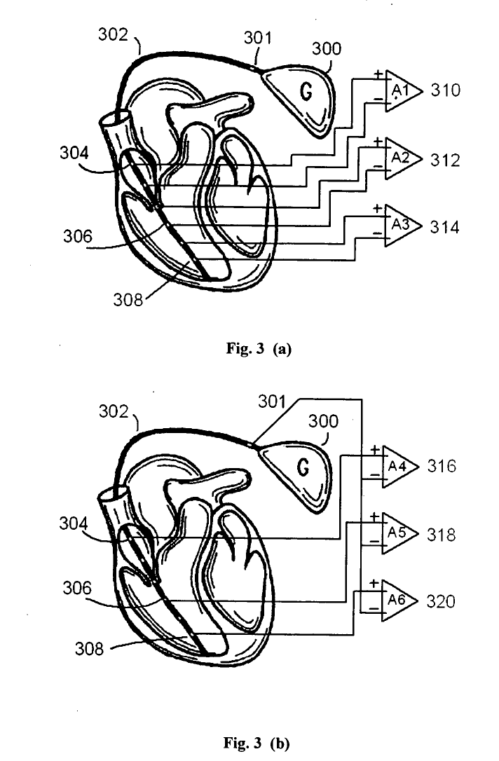

FIG. 3 shows an embodiment of an intra-cardiac lead system (bipolar and unipolar lead system), detecting vector system that can be used to detect cardiac ischemia. FIG. 3 (a) illustrates an Intra-cardiac bipolar lead system and FIG. 3 (b) illustrates an Intra-cardiac unipolar lead system. Block 300 may be one or more of a variety of sealed devices and 301 is the first sensor of the lead 302 which is utilized as a common reference electrode in bipolar measurement. There are different sensors in the lead which are implanted in the ventricular cavity. During intra-cardiac data acquisition, there are one or more different approaches, including but not limited to bipolar and unipolar lead system. 304, 306 and 308 are 3 different sensors in the lead system. In FIG. 3 (a), 310, 312 and 314 are 3 bipolar lead combinations for data acquisition. In FIG. 3 (b), 316, 318 and 320 are 3 unipolar leads. Unipolar leads may use the Wilson's center terminal (WCT) as the reference or common point. However, bipolar cardiac signals are preferably recorded by a close pair electrode (sensors, like 304, 306 and 308) comparing with a particular single electrode or sensor (like 301). With suitable choice of the reference position, the signal amplitude of the results and the sensitivity of the connection can be greatly affected.

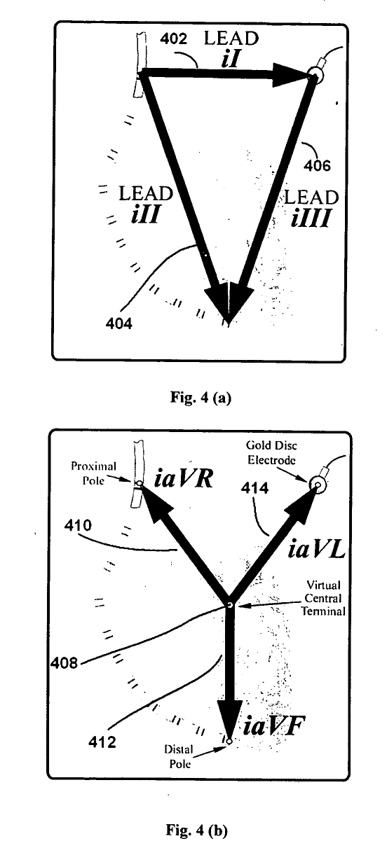

FIG. 4 (a) displays an embodiment including a three leads system for cardiac pathology detection. The wide bipolar lead configuration uses an indwelling catheter. The wide bipolar lead configuration also uses a subcutaneous disc electrode to simulate the implanted device can. The three electrodes form an internal Einthoven triangle. Leads iI (402), iII (404), and iIII (406) can thus be obtained and interpreted using traditional or novel ischemia detection algorithms. Based on this triangle vector system, more accurate information of MI/I pathology can be obtained. For example, in FIG. 4 (a), 404 lead II is more sensitive to the ischemia with right part of heart while 406 lead III is more sensitive to the left. Different lead systems can extract different information from the acquired data. The lead system employed in this embodiment is the result of the mapping from the surface lead system. Moreover, FIG. 4 (b) displays an Augmented Internal Leads (AIL) system, which can be developed to extract more information on MI/I pathologies. By comparing the proximal pole to 408 Virtual Central Terminal (VCT), a potential reference electrode which could be real central electrode and virtual potential estimation point, 410 Lead iaVR is generated. Comparing the distal pole to the 408 VCT generates 412 Lead iaVF. Finally, 414 Lead iaVL is generated by comparing the reference electrode (or the implanted devices can electrode) to 408 VCT. These leads, iaVR (410), iaVL (412), and iaVF (414), which can be recognized as internal mapping lead as aVR, aVL, and aVF in the surface lead system, provide valuable complementary data to aid in diagnosing ischemia. Compared with surface lead system, the lead definition in FIGS. 4 (a) and (b) enables the following intra-cardiac monitoring and analysis: cardiac mapping lead system (from whole body to heart), and virtual lead system which can extract new lead information from the physical electrode configuration). This can provide a more accurate approach to cardiac disease diagnosis and MI/I addressing.

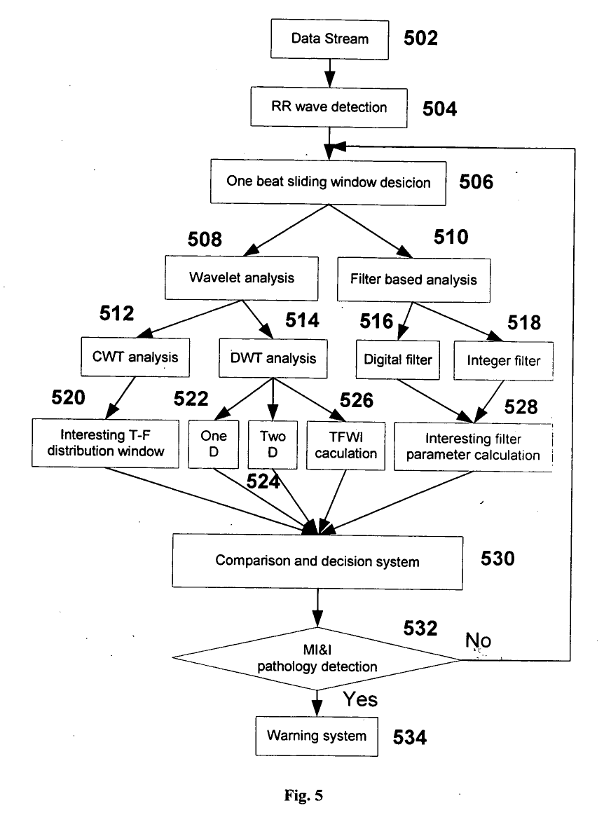

FIG. 5 illustrates an embodiment of the MI/I detection strategy. When a stream of data from one beat is inputted (block 502), a moving (sliding) window (block 506) is employed, which can be centered around R wave (block 504), for further pathology analysis. In order to be more flexible to different situations, the analysis window can be adjusted automatically in size and position. Block 508 is the wavelet analysis, a time frequency joint analysis, which is utilized to extract the signal changes in a certain time and frequency range. Block 510 is the filter based analysis through which the signal of interesting band width can be isolated for tracking the signal changes during ischemia events. In the block 508 wavelet analysis, either Continuous Wavelet Transform (CWT) 512 or Discrete Wavelet Transform (DWT) 514 or a combination of the two can be employed. For example, CWT is utilized for searching an interesting distribution window 520 which can discriminate the changes between normal and ischemic heart. At the same time, the block 520 also provides information for DWT analysis. After block 512 CWT analyses, more precise information and potential detection area for ischemia can be obtained. DWT may be computationally more reasonable for practical implementations in digital signal processor (DSP) and micro-processor. That is because DWT can be decomposed into limited parts with discrete numbers and values of enough accuracy while CWT needs significantly more calculation. DWT analysis 514 can be divided into two parts in an embodiment of the present invention: one-dimension (1D, wavelet spectrum analysis) 522 and two-dimension (2D, Frequency and time distribution) 524 analyses. Although not shown, for DWT analysis, 3 dimensional analyses (3D, energy distribution with frequency and time domain) can be further developed. Certain preferred embodiments may also include integer coefficient filter based wavelet analysis and calculation for ischemia event detection. Block 526 is an integrated wavelet index for time-frequency analysis (TFWI, time-frequency wavelet index).

Pacemakers or other implanted system have limited computational ability. Integer arithmetic based algorithm can be more proper and suitable. Module 510 may include different kinds signal filters, such as analog and digital filter, etc. Digital filter 516 and integer filter 518 are the extension and simplification for a preferred embodiment of wavelet analysis. In the module of integer filter 518, there are many choices of numerator for constructing a desired integer filter for ischemia event detection, such as filter order, etc. In certain embodiments, the choices for numerator are either (1−z^{-m}) or (1+z^{-m}), which determines where a zero of the digital filter is placed. In certain embodiments, the best choice is the one that places a zero where signal attenuation is needed with a reasonable value of m to further enhance the attenuation or further define other zero locations. The number of zeros chosen depends on the acceptable nominal bandwidth requirements. In one preferred embodiment, a digital filter structure includes a low pass filter having a notch at 60 Hz and a band pass filter which can amplify signals in a frequency range from 25-40 Hz and has a notch at 60 Hz. The filter has recursive structure and uses integer coefficients to simplify and speed up the calculations. Block 528 is a calculating function part of filter analysis which can adaptively choose the interesting part of the cardiac data, e.g. 25-40 Hz signal. Block 530 is the comparison function part which can accomplish calculations, parameter adjustment and index analysis for both baseline and clinical data. Block 532 is employed for the MI/I detection strategy, which depends on a variety of factors, such as patient status, environment, etc. Then, finding something wrong, such as detecting the MI/I, the strategy gives warning and indication to the patient or the doctor. Block 534 is a warning system.

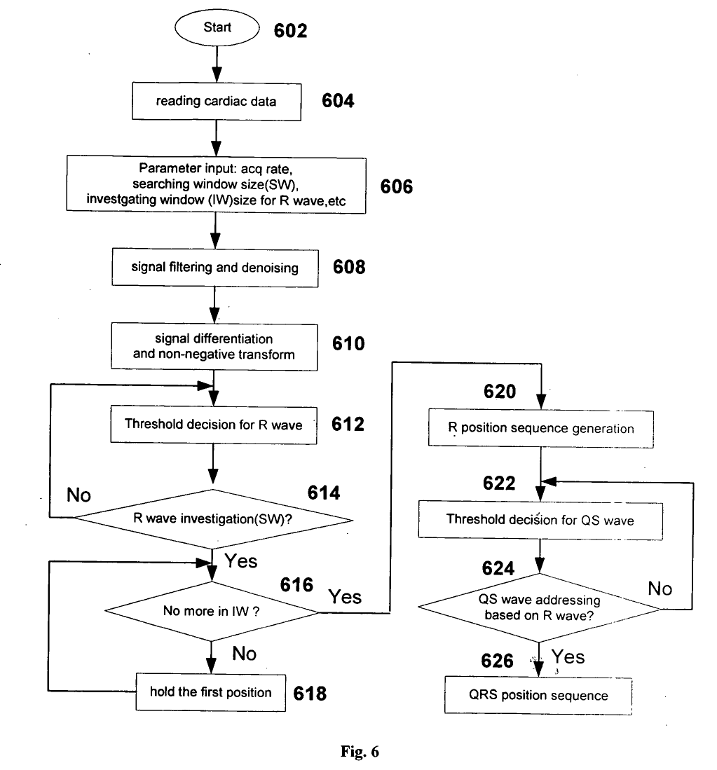

FIG. 6 illustrates an embodiment of a flow chart for QRS wave detection strategy. QRS wave detection is used for ischemia analysis because the cardiac ischemic information is within the QRS wave and ST segment. If there is certain occlusion or blockage of blood in a coronary vessel, the cells in the myocardium will be affected, resulting in changes in transmembrane ion channels and ion pumps, consequently affecting the cardiac action potential (AP). Changes in AP would include flow, which would result in changes in cardiac action potentials (APs), such as rate of depolarization, duration and rate of repolarization, and other altered morphologies such as early and delayed after depolarization. The MI/I changes result in changes in electrophysiological conduction in the heart as well. Consequently, these changes in APs and conduction cause changes in the entire QRS-T signal complex of the electrocardiogram signal recorded on the surface of the myocardium and the projected field potentials within the heart and inside and outside the torso. The changes in the electrocardiogram signals inside or outside the heart or inside the torso are picked up by leads placed inside or outside the heart. Corresponding changes in the entire QRS-T complex on the torso, outside the body, are picked up by the surface ECG recordings. In terms of the relationship between APs and ECG signal, the ECG, especially the QRS complex (depolarization), will be altered and at the same time the ST segment will be altered. Based on this knowledge, the a QRS analysis process may be detailed below.

Block 604 is a data reading function which acquires and transfers the cardiac data into the buffer or memory. Block 606 is a system parameter initialization. In a preferred embodiment, module 606 may include system data acquisition rate, searching window (SW) size for heart beat detection, investigating window (IW) size for R wave characterization, etc. Block 608 is a preliminary denoising and artifact rejection to increase the SNR for more accurate and reliable QRS detection and analysis. At the same time, module 608 can help to achieve more reliable ST segment signal which is prone to noise, such as body movement, etc. Blocks 610 and 612 are utilized for R wave detection and heart beat characterization. Block 610 employs signal differentiation to detect rapid changes in the fast portion of the cardiac signal, including the QRS complex. Block 612 represents the threshold decision to detect changes in the QRS indications of MI/I on different occasions. Certain preferred embodiments may include an adaptive threshold adjusting and estimating system. Block 614 is a decision function to determine whether an R wave is detected or not. When block 614 finds an R wave, the whole algorithm continues to execute block 616 to make sure that there is no incorrect R wave near the R wave it previously found. Then block 618 remembers the R wave position (time address). With block 618, a sequence can be generated for R wave position which will be utilized in the QRS energy calculation and estimation for detecting ischemia events. This is useful for subsequent CWT, DWT and TFWI analysis. After R wave addressing, the position of Q and S wave needs to be detected for different usages, such as ST-segment or T wave analysis. Block 622 is a threshold decision function for characterizing the Q and S wave. Then, block 624 is used to detect the Q and S wave which is based on the R wave addressing in the block 620. After block 624, the QRS position information and valid QRS sequence is obtained in block 626. FIG. 6 illustrates a process for QRS wave characterization.

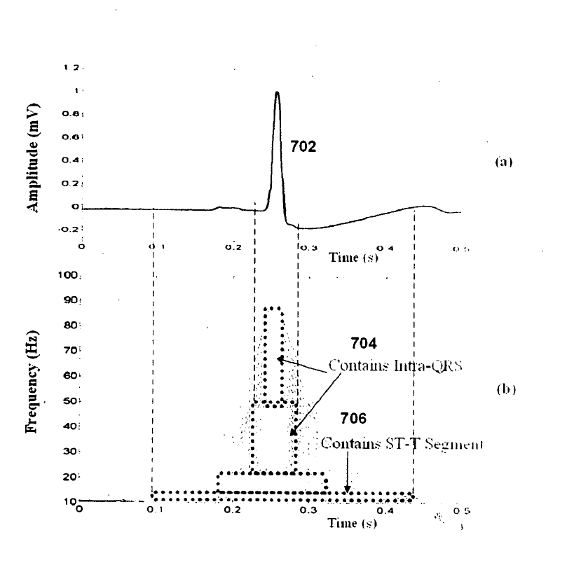

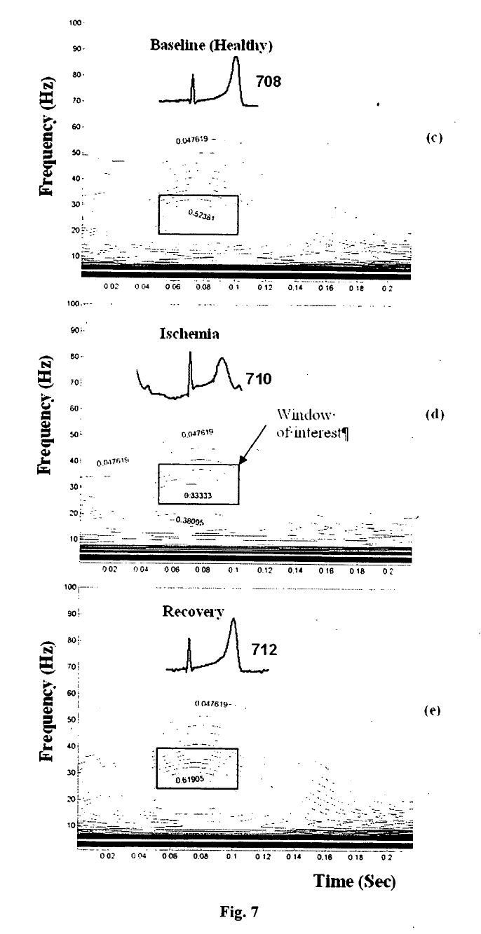

FIGS. 7(a) and 7(b) display one beat QRS complex 702 and the corresponding CWT analysis. FIG. 7(b) shows a window of interest (WOI) in cardiac data analysis for one beat, such as the intra-QRS and the ST-T segment. During the ischemia events, the time frequency distribution of the intra-QRS signal 704 and ST-T segment 706 will change, which can be utilized to monitor and track the ischemia events. FIG. 7(b) shows the time-frequency distribution, which also reflects the energy distribution during depolarization and repolarization of the beat. FIGS. 7(c) 708, 7(d) 710, and 7(e) 712, show the cardiac signal of single beat and corresponding 2-dimensional time-frequency distributions, which clearly detail the signal and time-frequency changes before, during and after an ischemia event. The time-frequency distribution along with the window of interest shows the WOI for ischemia analysis, respectively.

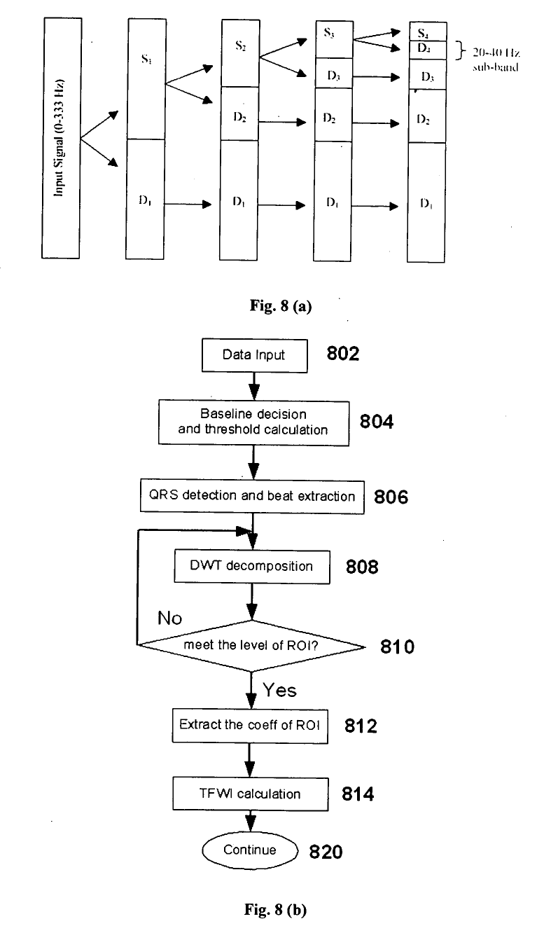

FIGS. 8(a) and 8(b) show an embodiment of the DWT decomposition strategy and the TFWI algorithm, respectively. FIG. 8(a) explains the frequency decomposition by the method of wavelet transform. In a preferred embodiment, the data acquisition rate is 666 Hz, which means the highest frequency of the digitized signal is 333 Hz. In level 4 of the wavelet decomposition, D4 corresponds to the 25-40 Hz range and the wavelet index of this range will be employed in the calculation and estimation of MI/I. FIG. 8(b) shows the calculation and strategy of the TFWI algorithm. Block 802 is the input of the intra-cardiac data. Block 804 is the baseline and threshold calculation and decision, which is utilized for comparison and detection of the cardiac pathologies. Block 806 is for QRS detection and addressing. After QRS extraction of each heart beat, an adaptive or fixed time window is employed for DWT decomposition 808. Block 810 is the judgment function to decide if the DWT decomposition has met the interesting level of time-frequency distribution; if not, the DWT procedure continues; and goes to next operation for signal processing. Block 812 is utilized for extracting corresponding time-frequency index for WOI of each heart beat. Then, Block 814 is for the calculation of TFWI. Block 820 is the end of the TFWI calculation function. One embodiment of TFWI, as implemented in this embodiment, can be seen in the following equation:

$${TFWI}_{{w}^{*} , {t}^{*}} = \frac{{\sum }_{{w}^{*}} {\sum }_{{t}^{*}} CWT ( w , t )}{\vert {t}^{*} \vert}$$

The embodiment calculates the signal in the small box of time-frequency space and calls it the Time-Frequency Window Index (TFWI). TFWI provides a simple energy analog for analysis. The essential idea in this embodiment is to sum up the signal energy within a frequency band of interest and the time band of interest. One particular frequency band of interest for this embodiment is found to be approximately within the range of 25-40 Hz.

The DWT in this embodiment allows for the implementation of wavelet transforms in the form of filter banks. Each filter bank preferably includes a low pass and a high pass filter succeeded by down-sampling by two. A number of filter banks are cascaded to achieve a multi-resolution wavelet analysis. This decomposition method implemented in the present embodiment acts as a sieve and separates the desired frequency sub-band. S is the low pass component of the input signal and D is the high pass component. The filter banks used for the decomposition in this embodiment are perfect reconstruction half band filters and the bandwidth of the signal in each block is dependent on the bandwidth of the input signal (approximately 0-400 Hz). At each step of the decomposition, the signal gets down sampled by 2 and the high and low frequency components get divided into separate bands. These filter banks, as implemented in this preferred embodiment, are a promising technique for signal decomposition and analysis for implantable devices.

### EXAMPLE 1

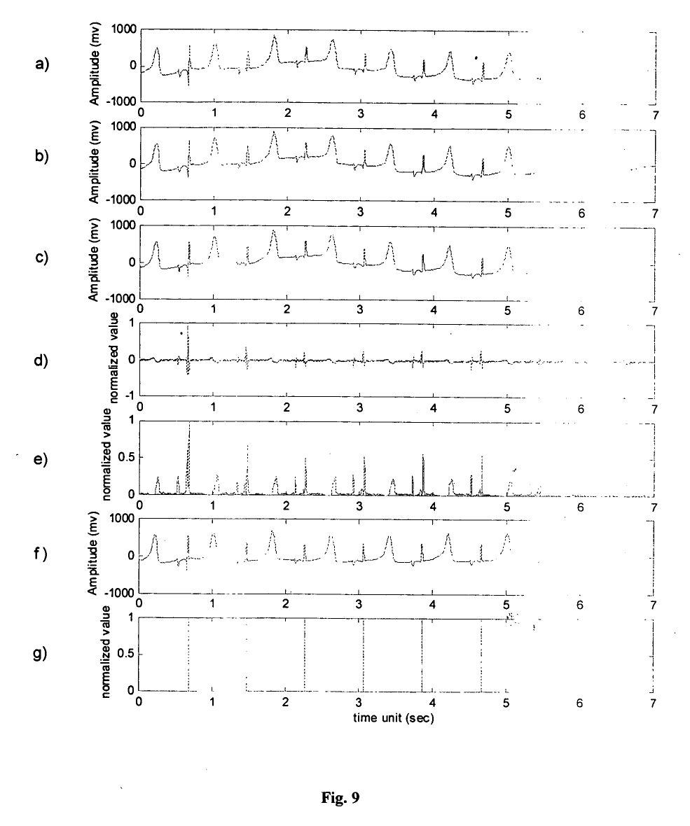

This example is to demonstrate a working sequence and detection strategy in accordance with an embodiment of the present invention. FIG. 9 shows QRS wave detection and addressing for one electrocardiogram signal from a normal heart;

The QRS detection strategy of this embodiment may preferably include 7 operations:
Operation 1: Reading the raw data from the buffer or hardware memory: This raw data has been filtered by the hardware pre-system and digitized by the acquisition system. (See a) FIG. 9.)
Operation 2: Intra-cardiac data normalization (See b) FIG. 9.): This operation is used to delete the static energy (average) from the signal. This operation can be used in an adaptive way to accommodate the baseline in different situation.

$$normalized\_signal ( i ) = signal ( i ) = signal ( i ) - {\sum }_{i = 1}^{n} {x}_{i}$$
Where signal is the raw signal of operation 1; n is determined by the user and is utilized for baseline analysis and decision;
Operation 3: Low pass filter: This operation is for the denoising of high frequency. The system acquisition rate is 666 Hz, so the highest Nyuquist frequency of the signal should be less than 333 Hz. Since an interesting part of detecting ischemia is focusing on 25-40 Hz, signal of high frequency is not very significant. For example, in this embodiment, the threshold frequency of the low pass filter 70 Hz. (See c) FIG. 9.)
Operation 4: Differentiation procedure: In order to accurately address the QRS waves in the data stream, this embodiment employs differentiation function within the algorithm. This is because the QRS wave is the fastest changing part of the data stream. From the fourth subplot, R wave has the biggest value in the differentiation data stream. (See d) FIG. 9.)
   differential_data(i)=xᵢ₊₁−xᵢ
Operation 5: Non-negative transformation (See e) FIG. 9.): After differentiation procedure, R wave can be found in the differential data stream. But an automatic and stable algorithm is developed for R wave detection. Non-negative transformation is utilized to enhance the R wave (those points which have the largest acceleration). Secondly, an adaptive threshold (the threshold can be learned in the data processing) is also developed to detect the R wave position. This embodiment uses 0.6 as the threshold (60% of the largest value as the threshold) in the normalized Non-negative data stream to address the R wave position.
Operation 6: Raw data cleaning and artifact rejection: In practice, there are some artifacts and low frequency noise, such as respiration, as well as high frequency (in operation 3). So in order to make the cardiac data cleaner for ischemia detection, this embodiment utilizes a high pass filter (for example, the threshold is 5 Hz). (See f) FIG. 9.)
Operation 7: R wave addressing and R pulse generation (See g) FIG. 9.). After the operations 1 to 6, clean and stable detection for R waves is obtained and a sequence for R waves can be generated as a result. The R pulse is used as a sequence for addressing the analysis window for ischemia detection. Based on R wave addressing, Q and S wave can be easily found which are used for the QRS feature analysis in MI/I detection.

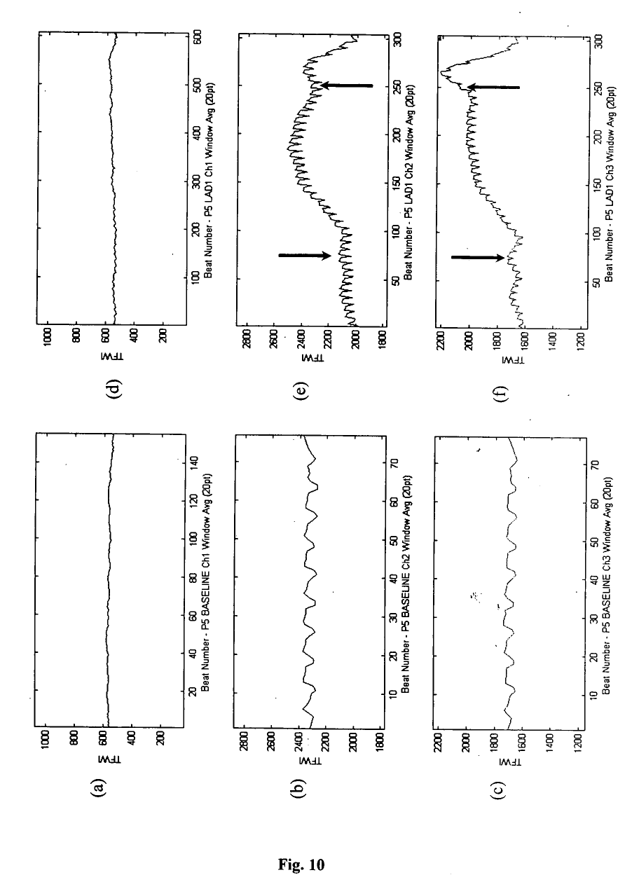

FIG. 10 illustrates the wavelet index, TFWI, calculation of 3 lead intra-cardiac systems, iI, iII, and iIII, as described in FIG. 4. When there is ischemia (for example, occlusion of left anterior descending artery, LAD, occlusion induces ischemia), the TFWI increases. During the perfusion phase, TFWI decreases and recovers towards the normal value. In FIG. 10, a), b) and c) are the time frequency window index (TFWI) calculation of baseline signal without any ischemia events in lead iI, iII, and iIII respectively. The d), e) and f) show demonstrate the TFWI calculation changes during the ischemia for the 3 leads.

Based on the QRS addressing and position sequence, a preferred embodiment employs a 400-point window which is centered on the R wave. In this window, wavelet based time-frequency analysis is utilized in to track the changes in the case of cardiac ischemia. FIG. 10 is the 25-40 Hz coefficients (TFWI) changes. The occlusion point can be clearly investigated which induces the cardiac ischemia and the perfusion point from which the TFWI index recovers back to normal range.

### EXAMPLE 2

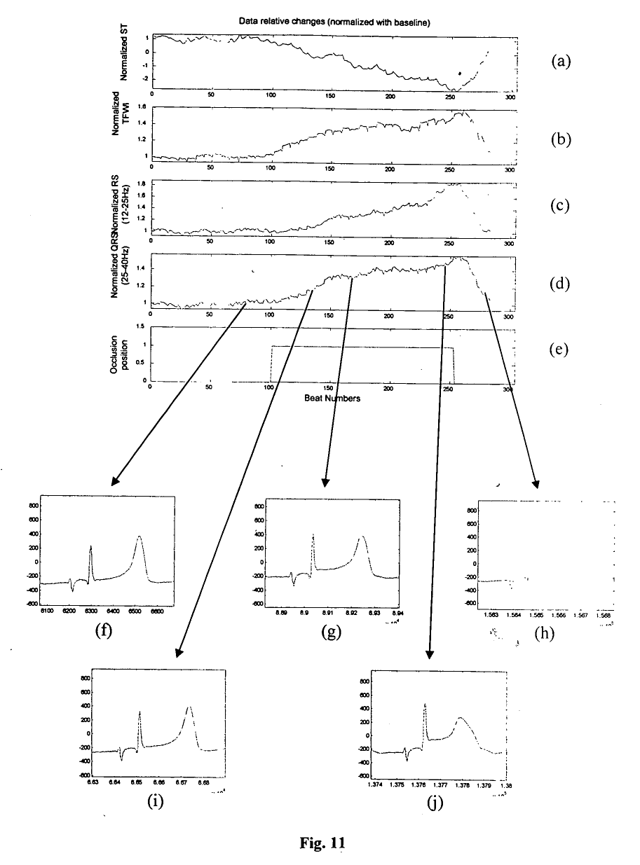

The second example includes an integer filter based ischemia event detection with a lower computational load requirement in accordance with an embodiment of the present invention. FIG. 11 demonstrates four calculation results for QRS analysis and ischemia event detection of: (1) intra-ST elevation, subplot a) in FIG. 10; (2) TFWI based QRS energy analysis, subplot b) in FIG. 10; (3) intra-QRS energy (12-25 Hz), subplot c) in FIG. 10, and (4) intra-QRS energy (25-40 Hz), subplot d) in FIG. 10. In FIG. 10, e) shows the occlusion time of ischemia event, from beat 100 to beat 255. It should be emphasized that the intra-QRS energy (12-25 Hz) and intra-QRS energy (25-40 Hz) are all calculated based on the integer filter, which will have a much lower calculation burden and thus is suitable for implementation in implantable devices with limited computing resources.

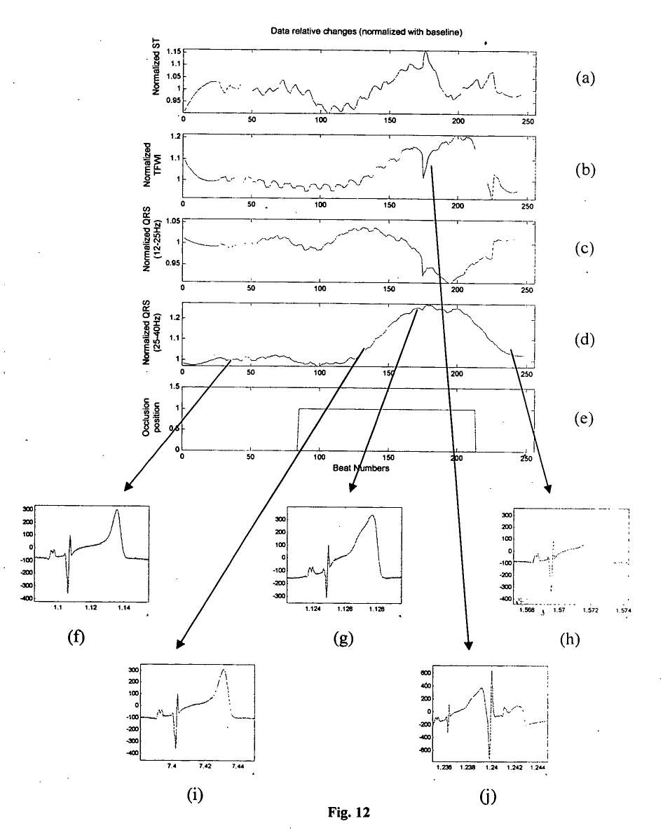

During cardiac occlusion,
1. Intra-ST segment may increase a much as 200%, but the absolute value of ST segment change is very small (30-150 microv) which may not be enough for reliable ischemia identification. And in case of some kinds of noisy situations, ST segment may be distorted greatly. Moreover, in real pacemaker devices, there is no standard ST segment and that is main reason to develop QRS energy to detect ischemia. (See a) in FIG. 10)
2. TFWI is the energy calculation based on wavelet analysis (See b) in FIG. 10). It needs more calculations to implement. See example 1 for additional details.
3. Intra-QRS energy (12-25 Hz) is mainly used to capture QRS energy in the low frequency band. The intra-QRS band (12-25 Hz) demonstrates the usual QRS energy distribution and changes during of MI/I events. In most MI/I events (LAD and circumflex occlusion), the main energy changes may be other than this band (in FIG. 11, intra-QRS energy (12-25 Hz) in subplot c), has the same trend with intra-QRS energy (25-40 Hz) in subplot d). But in FIG. 12(c), intra-QRS energy (12-25 Hz) does not show the QRS energy changes occurring during ischemia.) The differences in the (12-25 Hz) and (25-40 Hz) can also be utilized for MI/I detection and event characterization.
4. Intra-QRS energy (25-40 Hz) is utilized as a standard for MI/I detection. FIG. 11 and FIG. 12 demonstrate that intra-QRS energy (25-40 Hz, subplot d)) based MI/I detection is stable and accurate for either case.
5. The bottom subplot e) is the cardiac occlusion time.

In FIG. 11, f), i), g), j), and h) show the cardiac signal of single heart beat at the time of no ischemia, early ischemia, mid-ischemia, later ischemia, and acute recovery respectively.

FIG. 12 is an example for ischemia detection to illustrate that the energy of the intra-QRS (25-40 Hz) may provide better performance and stability than ST analysis. In this ischemia case, there is a noisy cycle near the 175th beat. This unexpected noisy beat changes the energy distribution. The noise effect can be seen from the first 3 subplots (FIG. 12(a) ST segment elevation, FIG. 12(b) TFWI energy and FIG. 12(c) intra-QRS energy (12-25 Hz).) However, intra-QRS energy (25-40 Hz) is free from this unexpected noise change, FIG. 12(d). Example 2 shows the feasibility and stability of integer filter based cardiac ischemia detection. FIG. 12(e) shows the cardiac occlusion time. In FIG. 12, f), i), g), j), and h) show the cardiac signal of single heart beat at the time of no ischemia, early ischemia, mid-ischemia, later ischemia, and acute recovery respectively.

It is, of course, understood that modifications of the present invention, in its various aspects, will be apparent to those skilled in the art. Additional method and device embodiments are possible, their specific features depending upon the particular application.

What is claimed:

1. A method of monitoring the heart of a subject for evidence of at least one of myocardial ischemia and infarction (MI/I) comprising:
sensing an intra-cardiac electrical signal from at least one lead positioned in a subject;
detecting MI/I using at least one of the QRS portion of the intra-cardiac electrical signal and the ST portion of the intra-cardiac electrical signal;
wherein the detecting MI/I includes using an integer coefficient filter to extract MI/I information from the intra-cardiac electrical signal.

2. A method as in claim 1, wherein sensing and the detecting are carried out using a device implanted inside a subject.

3. A method as in claim 2, wherein the method further comprises alerting the subject upon detection of MI/I using a signal from a device implanted in the subject.

4. A method as in claim 1, further comprising converting intra-cardiac signal to a digital value for MI/I analysis prior to the using an integer coefficient filter.

5. A method as in claim 1, wherein the integer coefficient filter is a filter selected from the group consisting of a hardware filter and a software filter.

6. A method as in claim 1, wherein the detecting MI/I uses the QRS portion of the intra-cardiac electrical signal, and wherein the intra-cardiac signal is at least one of a unipolar signal and a bipolar signal.

7. A method as in claim 1, wherein the detecting MI/I uses the ST portion of the intra-cardiac electrical signal, and wherein the intra-cardiac signal is at least one of a unipolar signal and a bipolar signal.

8. A method as in claim 1, wherein using the integer coefficient filter to extract MI/I information includes comparing a baseline signal to the intra-cardiac electrical signal at a frequency bandwith of 20-40 Hz.

9. A method of monitoring the heart for evidence of myocardial ischemia and infarction (MI/I) comprising:
sensing an intra-cardiac electrical signal from at least one lead positioned in a subject;
detecting MI/I using at least one of the QRS portion of the intra-cardiac electrical signal and the ST portion of the intra-cardiac electrical signal;
wherein the detecting MI/I includes using a quantized coefficient filter to extract MI/I information from the intra-cardiac electrical signal.

10. A method as in claim 9, wherein sensing and the detecting are carried out using a device implanted inside a subject.

11. A method as in claim 10, wherein the method further comprises alerting the subject upon detection of MI/I using a signal from a device implanted in the subject.

12. A method as in claim 9, further comprising converting intra-cardiac signal to a digital value for MI/I analysis prior to the using an integer coefficient filter.

13. A method as in claim 9, wherein the integer coefficient filter is a filter selected from the group consisting of a hardware filter and a software filter.

14. A method as in claim 9, wherein the detecting MI/I uses the QRS portion of the intra-cardiac electrical signal, and wherein the intra-cardiac signal is at least one of a unipolar signal and a bipolar signal.

15. A method as in claim 9, wherein the detecting MI/I uses the ST portion of the intra-cardiac electrical signal, and wherein the intra-cardiac signal is at least one of a unipolar signal and a bipolar signal.

16. A method as in claim 9, wherein using the integer coefficient filter to extract MI/I information includes comparing a baseline signal to the intra-cardiac electrical signal at a frequency bandwith of 20-40 Hz.
